(19) United States
(12) Patent Application Publication
(10) Pub. No.: US 2009/0042843 A1
(43) Pub. Date: Feb. 12, 2009

(54) Inhalation Device Containing Plural Doses of a Pharmaceutical Composition

(75) Inventor: Heinz-Gerd Klaes, Gau-bickelheim (DE)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(73) Assignee: BOEHRINGER INGELHEIM INTERNATIONAL GMBH, Ingelheim Am Rhein (DE)

(21) Appl. No.: 11/912,089

(22) PCT Filed: Apr. 28, 2006
(86) PCT No.: PCT/EP2006/061947

§ 371 Date: Mar. 20, 2008

(30) Foreign Application Priority Data
May 4, 2005 (EP) 05009773.2
Jun. 21, 2005 (EP) 05105426.0

(51) Int. Cl. A61K 31/56; A61K 31/439; A61P 11/00; A61K 31/538

(52) U.S. Cl. 514/171; 514/291; 514/299; 514/230.5

(57) ABSTRACT

The invention relates to an inhalation device comprising plural of doses of a pharmaceutical composition in powder form, wherein the pharmaceutical composition comprises one or more, preferably one, anticholinergic, optionally in combination with a pharmaceutically acceptable excipient.

The invention relates to an inhalation device comprising plural doses of a pharmaceutical composition in powder form, wherein the pharmaceutical composition comprises one or more anticholinergic, optionally in combination with a pharmaceutically acceptable excipient.

### DESCRIPTION OF THE INVENTION

The invention relates to an inhalation device comprising plural of doses of a pharmaceutical composition in powder form, wherein the pharmaceutical composition comprises one or more, preferably one, anticholinergic, optionally in combination with a pharmaceutically acceptable excipient, wherein the anticholinergic is selected from the group consisting of
a) tiotropium salts,
b) compounds of formula 1c

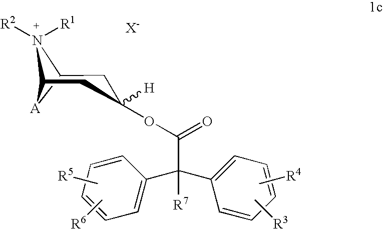

wherein
A denotes a double-bonded group selected from among

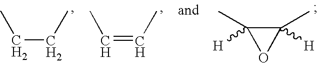

X⁻ denotes an anion with a single negative charge, preferably an anion selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹ and R² which may be identical or different denote a group selected from among methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine;
c) compounds of formula 1d

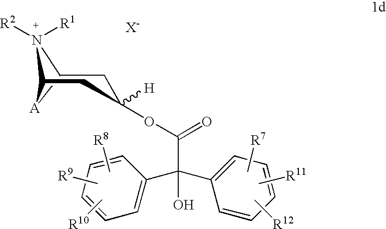

wherein
A, X⁻, R¹ and R² may have the meanings as mentioned hereinbefore and wherein
R⁷, R⁸, R⁹, R¹⁰, R¹¹ and R¹² , which may be identical or different, denote hydrogen, methyl, ethyl, methyloxy, ethyloxy, hydroxy, fluorine, chlorine, bromine, CN, CF₃ or NO₂, with the proviso that at least one of the groups R⁷, R⁸, R⁹, R¹⁰, R¹¹ and R¹² is not hydrogen,
d) compounds of formula 1e

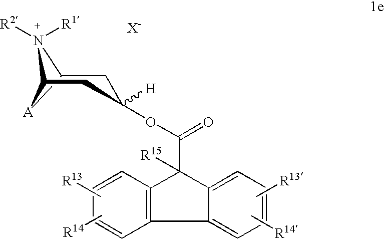

wherein A and X⁻ may have the meanings as mentioned hereinbefore, and wherein
R¹⁵ denotes hydrogen, hydroxy, methyl, ethyl, —CF₃, CHF₂ or fluorine;
R^{1′} and R^{2′} which may be identical or different denote C₁-C₅-alkyl which may optionally be substituted by C₃-C₆-cycloalkyl, hydroxy or halogen, or

R^{1′} and R^{2′} together denote a —C₃-C₅-alkylene-bridge;
R¹³, R¹⁴, R^{13′} and R^{14′} which may be identical or different denote hydrogen, —C₁-C₄-alkyl, —C₁-C₄-alkyloxy, hydroxy, —CF₃, —CHF₂, CN, NO₂ or halogen,
e) compounds of formula 1f

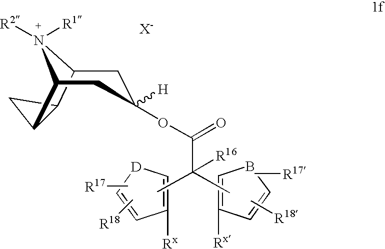

wherein X⁻ may have the meanings as mentioned hereinbefore, and wherein
D and B which may be identical or different, preferably identical, denote —O, —S, —NH, —CH₂, —CH═CH, or —N(C₁-C₄-alkyl)—;
R¹⁶ denotes hydrogen, hydroxy, —C₁-C₄-alkyl, —C₁-C₄-alkyloxy, —C₁-C₄-alkylene-Halogen, —O—C₁-C₄-alkylene-halogen, —C₁—C₄-alkylene-OH, —CF₃, CHF₂, —C₁—C₄-alkylene-C₁-C₄-alkyloxy, —O—COC₁—C₄-alkyl, —O—COC₁—C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or

R^{1″} and R^{2″} together denote a —C₃-C₅-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂ or halogen;
R^{x} and R^{x′} which may be identical or different, denote hydrogen, C₁-C₄-alkyl, C₁-C₄-alkyloxy, hydroxy, —CF₃, —CHF₂, CN, NO₂ or halogen or

R^{x} and R^{x′} together denote a single bond or a bridging group selected from among the bridges —O—, —S, —NH, —CH₂, —CH₂-CH₂—, —N(C₁-C₄-alkyl), —CH(C₁-C₄-alkyl)- and —C(C₁-C₄-alkyl)₂,
and
f) compounds of formula 1g

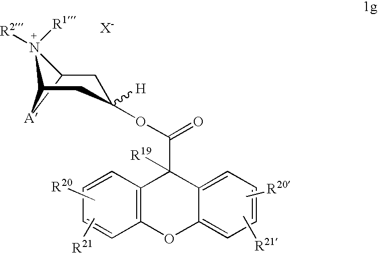

wherein X⁻ may have the meanings as mentioned hereinbefore, and wherein
A′ denotes a double-bonded group selected from among

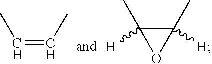

R¹⁹ denotes hydroxy, methyl, hydroxymethyl, ethyl, —CF₃, CHF₂ or fluorine;
R^{1′″} and R^{2′″} which may be identical or different denote C₁-C₅-alkyl which may optionally be substituted by C₃-C₆-cycloalkyl, hydroxy or halogen, or

R^{1′″} and R^{2′″} together denote a —C₃-C₅-alkylene-bridge;
R²⁰, R²¹, R^{20′} and R^{21′} which may be identical or different denote hydrogen, —C₁-C₄-alkyl, —C₁-C₄-alkyloxy, hydroxy, —CF₃, —CHF₂, CN, NO₂ or halogen.

The compounds of formula 1c are known in the art (WO 02/32899).

In a preferred embodiment of the invention the method comprises administration of compounds of formula 1c, wherein
X⁻ denotes bromide;
R¹ and R² which may be identical or different denote a group selected from methyl and ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine,
   optionally together with a pharmaceutically acceptable excipient.

Of particular importance within the method according to the invention are compounds of general formula 1c, wherein
A denotes a double-bonded group selected from among

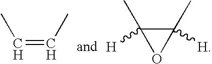

The compounds of formula 1c, may optionally be administered in the form of the individual optical isomers, mixtures of the individual enantiomers or racemates thereof.

Of particular importance within the method according to the invention are the following compounds of formula 1c:
tropenol 2,2-diphenylpropionic acid ester methobromide,
scopine 2,2-diphenylpropionic acid ester methobromide,
scopine 2-fluoro-2,2-diphenylacetic acid ester methobromide and
tropenol 2-fluoro-2,2-diphenylacetic acid ester methobromide.

The compounds of formula 1d are known in the art (WO 02/32898).

In a preferred embodiment of the invention the method comprises administration of compounds of formula 1d, wherein
A denotes a double-bonded group selected from among

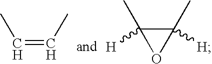

X⁻ denotes bromide;
R¹ and R² which may be identical or different denote methyl or ethyl, preferably methyl;
R⁷, R⁸, R⁹, R¹⁰, R¹¹ and R¹², which may be identical or different, denote hydrogen, fluorine, chlorine or bromine, preferably fluorine with the proviso that at least one of the groups R⁷, R⁸, R⁹, R¹⁰, R¹¹ and R¹² not hydrogen,
   optionally together with a pharmaceutically acceptable excipient.

Of particular importance within the method according to the invention are the following compounds of formula 1d:
tropenol 3,3′,4,4′-tetrafluorobenzilic acid ester methobromide,
scopine 3,3′,4,4′-tetrafluorobenzilic acid ester methobromide,
scopine 4,4′-difluorobenzilic acid ester methobromide,
tropenol 4,4′-difluorobenzilic acid ester methobromide,
scopine 3,3′-difluorobenzilic acid ester methobromide, and
tropenol 3,3′-difluorobenzilic acid ester methobromide.

The pharmaceutical compositions according to the invention may contain the compounds of formula 1d optionally in the form of the individual optical isomers, mixtures of the individual enantiomers or racemates thereof.

The compounds of formula 1e are known in the art (WO 03/064419).

In a preferred embodiment of the invention the method comprises administration of compounds of formula 1e, wherein
A denotes a double-bonded group selected from among

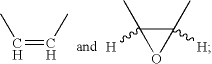

X⁻ denotes an anion selected from among chloride, bromide and methanesulphonate, preferably bromide;
R¹⁵ denotes hydroxy, methyl or fluorine, preferably methyl or hydroxy;
R^{1′} and R^{2′} which may be identical or different represent methyl or ethyl, preferably methyl;
R¹³, R¹⁴, R^{13′} and R^{14′} which may be identical or different represent hydrogen, —CF₃, —CHF₂ or fluorine, preferably hydrogen or fluorine,
   optionally together with a pharmaceutically acceptable excipient.

In another preferred embodiment of the invention the method comprises administration of compounds of formula 1e, wherein
A denotes a double-bonded group selected from among

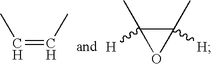

X⁻ denotes bromide;
R¹⁵ denotes hydroxy or methyl, preferably methyl;
R^{1′} and R^{2′} which may be identical or different represent methyl or ethyl, preferably methyl;
R¹³ , R¹⁴, R^{13′} and R^{14′} which may be identical or different represent hydrogen or fluorine, optionally together with a pharmaceutically acceptable excipient.

Of particular importance within the method according to the invention are the following compounds of formula 1e:
tropenol 9-hydroxy-fluorene-9-carboxylate methobromide ;
tropenol 9-fluoro-fluorene-9-carboxylate methobromide;
scopine 9-hydroxy-fluorene-9-carboxylate methobromide;
scopine 9-fluoro-fluorene-9-carboxylate methobromide;
tropenol 9-methyl-fluorene-9-carboxylate methobromide;
scopine 9-methyl-fluorene-9-carboxylate methobromide.

The pharmaceutical compositions according to the invention may contain the compounds of formula le optionally in the form of the individual optical isomers, mixtures of the individual enantiomers or racemates thereof.

The compounds of formula 1f are known in the art (WO 03/064418).

In another preferred embodiment of the invention the method comprises administration of compounds of formula 1f wherein
X⁻ denotes chloride, bromide, or methanesulphonate, preferably bromide;
D and B which may be identical or different, preferably identical, denote —O, —S, —NH or —CH═CH—;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine, or

R^{1″} and R^{2″} together denote a —C₃-C₄-alkylene-bridge;
R¹⁷, R¹⁸, R^{17′} and R^{18′}, which may be identical or different, denote hydrogen, C1-C4-alkyl, C1-C4-alkyloxy, hydroxy, —CF3, —CHF2, CN, NO2, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine or

R^{x} and R^{x′} together denote a single bond or a bridging group selected from among the bridges —O, —S, —NH— and —CH₂—,

optionally together with a pharmaceutically acceptable excipient.

In another preferred embodiment of the invention the method comprises administration of compounds of formula 1f, wherein
X⁻ denotes chloride, bromide, or methanesulphonate, preferably bromide;
D and B which may be identical or different, preferably identical, denote —S or —CH═CH—;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen or

R^{x} and R^{x′} together denote a single bond or the bridging group —O—,

optionally together with a pharmaceutically acceptable excipient.

In another preferred embodiment of the invention the method comprises administration of compounds of formula 1f, wherein
X⁻ denotes bromide;
D and B denote —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen or

R^{x} and R^{x′} together denote a single bond or the bridging group —O—,

optionally together with a pharmaceutically acceptable excipient.

Of particular importance within the method according to the invention are the following compounds of formula 1f:
cyclopropyltropine benzilate methobromide;
cyclopropyltropine 2,2-diphenylpropionate methobromide;
cyclopropyltropine 9-hydroxy-xanthene-9-carboxylate methobromide;
cyclopropyltropine 9-methyl-fluorene-9-carboxylate methobromide;
cyclopropyltropine 9-methyl-xanthene-9-carboxylate methobromide;
cyclopropyltropine 9-hydroxy-fluorene-9-carboxylate methobromide;
cyclopropyltropine methyl 4,4′-difluorobenzilate methobromide.

The pharmaceutical compositions according to the invention may contain the compounds of formula 1f optionally in the form of the individual optical isomers, mixtures of the individual enantiomers or racemates thereof.

The compounds of formula 1g are known in the art (WO 03/064417).

In another preferred embodiment of the invention the method comprises administration of compounds of formula 1g wherein
A′ denotes a double-bonded group selected from among

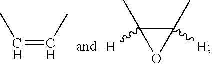

X⁻ denotes chloride, bromide or methanesulphonate, preferably bromide;
R¹⁹ denotes hydroxy or methyl;
R^{1′″} and R^{2′″} which may be identical or different represent methyl or ethyl, preferably methyl;
R²⁰, R²¹, R^{20′} and R^{21′} which may be identical or different represent hydrogen, —CF₃, —CHF₂ or fluorine, preferably hydrogen or fluorine,
   optionally together with a pharmaceutically acceptable excipient.

In another preferred embodiment of the invention the method comprises administration of compounds of formula 1g wherein
A′ denotes a double-bonded group selected from among

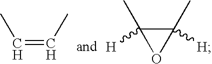

X⁻ denotes bromide;
R¹⁹ denotes hydroxy or methyl, preferably methyl;
R^{1′″} and R^{2′″} which may be identical or different represent methyl or ethyl, preferably methyl;
R³, R⁴, R^{3′} and R^{4′} which may be identical or different represent hydrogen or fluorine,
   optionally together with a pharmaceutically acceptable excipient.

Of particular importance within the method according to the invention are the following compounds of formula 1g:
tropenol 9-hydroxy-xanthene-9-carboxylate methobromide;
scopine 9-hydroxy-xanthene-9-carboxylate methobromide;
tropenol 9-methyl-xanthene-9-carboxylate methobromide;
scopine 9-methyl-xanthene-9-carboxylate methobromide;
tropenol 9-ethyl-xanthene-9-carboxylate methobromide;
tropenol 9-difluoromethyl-xanthene-9-carboxylate methobromide;
scopine 9-hydroxymethyl-xanthene-9-carboxylate methobromide

The pharmaceutical compositions according to the invention may contain the compounds of formula 1g optionally in the form of the individual optical isomers, mixtures of the individual enantiomers or racemates thereof.

The alkyl groups used, unless otherwise stated, are branched and unbranched alkyl groups having 1 to 5 carbon atoms. Examples include: methyl, ethyl, propyl or butyl. The groups methyl, ethyl, propyl or butyl may optionally also be referred to by the abbreviations Me, Et, Prop or Bu. Unless otherwise stated, the definitions propyl and butyl also include all possible isomeric forms of the groups in question. Thus, for example, propyl includes n-propyl and iso-propyl, butyl includes iso-butyl, sec. butyl and tert.-butyl, etc.

The cycloalkyl groups used, unless otherwise stated, are alicyclic groups with 3 to 6 carbon atoms. These are the cyclopropyl, cyclobutyl, cyclopentyl and cyclohexyl groups. According to the invention cyclopropyl is of particular importance within the scope of the present invention.

The alkylene groups used, unless otherwise stated, are branched and unbranched double-bonded alkyl bridges with 1 to 5 carbon atoms. Examples include: methylene, ethylene, propylene or butylene.

The alkylene-halogen groups used, unless otherwise stated, are branched and unbranched double-bonded alkyl bridges with 1 to 4 carbon atoms which may be mono-, di- or trisubstituted, preferably disubstituted, by a halogen. Accordingly, unless otherwise stated, the term alkylene-OH groups denotes branched and unbranched double-bonded alkyl bridges with 1 to 4 carbon atoms which may be mono-, di- or trisubstituted, preferably monosubstituted, by a hydroxy.

The alkyloxy groups used, unless otherwise stated, are branched and unbranched alkyl groups with 1 to 5 carbon atoms which are linked via an oxygen atom. The following may be mentioned, for example: methyloxy, ethyloxy, propyloxy or butyloxy. The groups methyloxy, ethyloxy, propyloxy or butyloxy may optionally also be referred to by the abbreviations MeO, EtO, PropO or BuO. Unless otherwise stated, the definitions propyloxy and butyloxy also include all possible isomeric forms of the groups in question. Thus, for example, propyloxy includes n-propyloxy and iso-propyloxy, butyloxy includes iso-butyloxy, sec. butyloxy and tert.-butyloxy, etc. The word alkoxy may also possibly be used within the scope of the present invention instead of the word alkyloxy. The groups methyloxy, ethyloxy, propyloxy or butyloxy may optionally also be referred to as methoxy, ethoxy, propoxy or butoxy.

The alkylene-alkyloxy groups used, unless otherwise stated, are branched and unbranched double-bonded alkyl bridges with 1 to 5 carbon atoms which may be mono-, di- or trisubstituted, preferably monosubstituted, by an alkyloxy group.

The —O—CO-alkyl groups used, unless otherwise stated, are branched and unbranched alkyl groups with 1 to 4 carbon atoms which are bonded via an ester group. The alkyl groups are bonded directly to the carbonylcarbon of the ester group. The term —O—CO-alkyl-halogen group should be understood analogously. The group —O—CO—CF₃ denotes trifluoroacetate.

Within the scope of the present invention halogen denotes fluorine, chlorine, bromine or iodine. Unless otherwise stated, fluorine and bromine are the preferred halogens. The group CO denotes a carbonyl group.

One aspect of the invention is directed to an inhalation device, in which the plural of doses are contained in one reservoir. In another aspect of the invention, the inhalation device comprises the plural of doses in a multi-dose blister pack. In another aspect of the invention the inhalation device comprises the multi-dose blister pack in form of a circular disc having a plurality of frangible containers arranged in a circle and containing medicament in powder form. In another aspect of the invention the inhalation device comprises the multi-dose blister pack in form of blister strip.

The inhalation device according to the invention comprises one or more anticholinergic compounds preferably in admixture with a pharmaceutically acceptable excipient to form a powder mixture. The following pharmaceutically acceptable excipients may be used to prepare these inhalable powder mixtures according to the invention: monosaccharides (e.g. glucose or arabinose), disaccharides (e.g. lactose, saccharose, maltose, trehalose), oligo- and polysaccharides (e.g. dextrane), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose and trehalose are the particularly preferred excipients, while lactose, preferably in form of its monohydrate is most particularly preferred.

The ancholinergics may be used in the form of their racemates, enantiomers or mixtures thereof. The separation of enantiomers from the racemates may be carried out using methods known in the art (e.g. by chromatography on chiral phases, etc.).

Optionally, the inhalation device according to the invention contains plural of doses of a medicament on powder form, that contains beside an ancholinergic another active ingredient.

Preferably the additional active ingredient is a beta₂ agonist which is selected from the group consisting of albuterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts and the hydrates thereof.

According to the instant invention more preferred beta₂ agonists are selected from the group consisting of bambuterol, bitolterol, carbuterol, clenbuterol, fenoterol, formoterol, hexoprenaline, ibuterol, pirbuterol, procaterol, reproterol, salmeterol, sulphonterol, terbutaline, tolubuterol, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1 2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4 -benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts and the hydrates thereof.

More preferably, the betamimetics used as within the compositions according to the invention are selected from among fenoterol, formoterol, salmeterol, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, optionally in the form of the racemates, the enantiomers, the diastereomers and optionally the pharmacologically acceptable acid addition salts thereof, and the hydrates thereof. Of the betamimetics mentioned above the compounds formoterol, salmeterol, 3-(4-{6-[2-Hydroxy-2-(4-hydroxy-3-hydroxymethyl-phenyl)-ethylamino]-hexyloxy}-butyl)-benzenesulfoneamide, and 5-[2-(5,6-Diethyl-indan-2-ylamino)-1-hydroxy-ethyl]-8-hydroxy-1H-quinolin-2-one are particularly preferred, optionally in the form of the racemates, the enantiomers, the diastereomers and optionally the pharmacologically acceptable acid addition salts thereof, and the hydrates thereof. Of the betamimetics mentioned above the compounds formoterol and salmeterol are particularly preferred, optionally in the form of the racemates, the enantiomers, the diastereomers and optionally the pharmacologically acceptable acid addition salts thereof, and the hydrates thereof. It will be apparent to those skilled in the art the term betamimetics is used interchangeably with beta₂ agonists herein.

Examples of pharmacologically acceptable acid addition salts of the betamimetics according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2.4-difluorophenyl)salicylic acid or maleic acid. If desired, mixtures of the abovementioned acids may also be used to prepare the salts of the betamimetics.

According to the invention, the salts of the betamimetics selected from among the hydrochloride, hydrobromide, sulphate, phosphate, fumarate, methanesulphonate, 4-phenylcinnamate, 5-(2.4-difluorophenyl)salicylate, maleate and xinafoate are preferred. Particularly preferred are the salts of in the case of salmeterol selected from among the hydrochloride, sulphate, 4-phenylcinnamate, 5-(2.4-difluorophenyl)salicylate and xinafoate, of which the 4-phenylcinnamate, 5-(2.4-difluorophenyl)salicylate and especially xinafoate are particularly important. Particularly preferred are the salts of in the case of formoterol selected from the hydrochloride, sulphate and fumarate, of which the hydrochloride and fumarate are particularly preferred. Of exceptional importance according to the invention is formoterol fumarate.

Salts of salmeterol, formoterol, 3-(4-{6-[2-Hydroxy-2-(4-hydroxy-3-hydroxymethyl-phenyl)-ethylamino]-hexyloxy}-butyl)-benzenesulfoneamide, and 5-[2-(5,6-Diethyl-indan-2-ylamino)-1-hydroxy-ethyl]-8-hydroxy-1H-quinolin-2-one, are preferably used as the betamimetics according to the invention. Of particular importance according to the invention are salmeterol and formoterol salts. Any reference to the term betamimetics also includes a reference to the relevant enantiomers or mixtures thereof.

In the pharmaceutical compositions according to the invention, the betamimetics may be present in the form of their racemates, enantiomers or mixtures thereof. The separation of the enantiomers from the racemates may be carried out using methods known in the art (e.g. by chromatography on chiral phases, etc.) If the betamimetics are used in the form of their enantiomers, it is particularly preferable to use the enantiomers in the R configuration at the C—OH group.

Optionally, the inhalation device according to the invention contains plural of doses of a medicament in powder form, that contains beside one ancholinergic a steroid as another active ingredient.

In such medicament combinations the steroid is preferably selected from among prednisolone, prednisone, butixocortpropionate, RPR-106541, flunisolide, beclomethasone, triamcinolone, budesonide, fluticasone, mometasone, ciclesonide, rofleponide, ST-126 , dexamethasone, (S)-fluoromethyl 6α,9α-difluoro-17α-[(2-furanylcarbonyl) oxy]-11β-hydroxy-16α-methyl-3-oxo-androsta-1,4-diene-17β-carbothionate, (S)-(2-oxo-tetrahydro-furan-3S-yl)6α,9α-difluoro-11β-hydroxy-16α-methyl-3-oxo-17α-propionyloxy-androsta-1,4-diene-17β-carbothionate, and etiprednol-dichloroacetate (BNP-166), optionally in the form of the racemates, enantiomers or diastereomers thereof and optionally in the form of the salts and derivatives thereof, the solvates and/or hydrates thereof.

In particularly preferred medicament combinations the steroid is selected from the group comprising flunisolide , beclomethasone , triamcinolone , budesonide, fluticasone, mometasone, ciclesonide, rofleponide, ST-126, dexamethasone, (S)-fluoromethyl 6α,9α-difluoro-17α-[(2-furanylcarbonyl)oxy]-11β-hydroxy-16α-methyl-3-oxo-androsta-1,4-diene-17β-carbothionate, (S)-(2-oxo-tetrahydro-furan-3S-yl)6α,9α-difluoro-11β-hydroxy-16α-methyl-3-oxo-17α-propionyloxy-androsta-1,4-diene-17β-carbothionate, and etiprednol-dichloroacetate, optionally in the form of the racemates, enantiomers or diastereomers thereof and optionally in the form of the salts and derivatives thereof, the solvates and/or hydrates thereof.

In particularly preferred medicament combinations the steroid is selected from the group comprising budesonide, fluticasone, mometasone, ciclesonide, (S)-fluoromethyl 6α,9 α-difluoro-17α-[(2-furanylcarbonyl)oxy]-11β-hydroxy-16α-methyl-3-oxo-androsta-1,4-diene-17β-carbothionate, and etiprednol-dichloroacetate, optionally in the form of the racemates, enantiomers or diastereomers thereof and optionally in the form of the salts and derivatives thereof, the solvates and/or hydrates thereof.

Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, propionates, dihydrogen phosphates, palmitates, pivalates or furoates.

Optionally, the inhalation device according to the invention contains plural of doses of a medicament on powder form, that contains beside one anticholinergic additionally both, one of the betamimetics mentioned hereinbefore and one of the steroids mentioned hereinbefore.

In another preferred embodiment according to the invention the inhalation device according to U.S. Pat. No. 5,590,645 is applied. The disclosure of U.S. Pat. No. 5,590,645 is incorporated herein by reference in its entirety. U.S. Pat. No. 5,590,645 describes an inhalation device for use with a medicament pack in which at least one container for a pharmaceutical composition in powder form is defined between two sheets peelably secured to one another. The device comprises means for peeling the sheets apart at an opening station to open the container; and an outlet, communicating with the opened container, through which a user can inhale medicament in powder form from the opened container.

Accordingly, in a preferred embodiment the invention relates to an inhalation device comprising a medicament pack having a plurality of containers for containing a pharmaceutical composition in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale the pharmaceutical composition in powder form from such an opened container, and indexing means for indexing in communication with said outlet containers of a medicament pack in use with said inhalation device, wherein the pharmaceutical composition comprises one or more, preferably one, anticholinergic.

In another preferred embodiment according to the invention the inhalation device according to U.S. Pat. No. 4,627,432 is applied. The disclosure of U.S. Pat. No. 4,627,432 is incorporated herein by reference in its entirety. U.S. Pat. No. 4627432 describes a device for administering medicaments to patients which comprises a housing containing a cylindrical chamber. A support is arranged inside the chamber to support a carrier, such as a blister pack. The blister pack has a plurality of containers or blisters arranged in a circle. When a blister pack is located on the support its blisters are located in holes in the support member. A plunger is arranged to enter the chamber through a hole to engage and open a blister registered with it. When the blister is opened, medicament can be withdrawn by a patient inhaling through a mouthpiece. An external member is provided to rotate the support member to register the blister with the plunger in turn. Air can conveniently enter the chamber through a hole in a cover which is removable to permit blister packs to be loaded into the chamber onto the support member.

Accordingly, in another preferred embodiment the invention relates to an inhalation device, in which the pharmaceutical composition in powder form is contained in a plurality of containers arranged in a circle on a carrier, the said inhalation device being further characterised by a housing with a chamber therein, an air inlet into the chamber, a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the pharmaceutical composition will be released from the container and entrained in the air flow produced by a patient inhaling through the outlet, and means between said disc and said housing for rotatably indexing the disc to register each of the apertures in turn with the housing opening, wherein the pharmaceutical composition comprises one or more, preferably one, anticholinergic.

In another preferred embodiment according to the invention the inhalation device according to WO 95/16483 is applied. The disclosure of WO 95/16483 is incorporated herein by reference in its entirety. WO 95/16483 describes an inhalation device for dispensing doses of a pharmaceutical composition in powder form that comprises a housing which houses a cylindrical container. The container has a number of helically arranged compartments each of which contains a respective dose of the pharmaceutical composition. In order to dispense the pharmaceutical composition from a compartment, that compartment is moved into registry with an airway in the device by means of an indexing mechanism, and the user sucks on a mouthpiece on the housing, which mouthpiece communicates with an air inlet via the airway. The flow of air through the airway ejects the dose of material. The container can constitute a replaceable cartridge.

Accordingly, in another preferred embodiment the invention relates to an inhalation device for dispensing single doses of a pharmaceutical composition in powder form, the device comprising a housing carrying a mouthpiece which communicates with an air inlet through an airway within the housing, a cylindrical container contained within the housing, the container having a plurality of compartments therein, each compartment containing a respective dose of the pharmaceutical composition, operating means for moving the container relative to the airway so as to bring successive compartments into registry with the airway and enable the doses of the pharmaceutical composition to be discharged therefrom, wherein the compartments are angularly and axially spaced relative to each other so as to define a helical path which is substantially coaxial with the axis of the container, wherein the pharmaceutical composition comprises one or more, preferably one, anticholinergic.

In another preferred embodiment according to the invention the inhalation device according to WO 95/31238 is applied. The disclosure of WO 95/31238 is incorporated herein by reference in its entirety. WO 95/31238 describes an inhalation device for dispensing single doses of pharmaceutical composition in powder form which has a housing for holding a container which has a number of sealed apertures containing individual encapsulating doses of medicament. The container can move relative to the housing to allow each aperture in succession to be brought into registry with an airway which communicates with a mouthpiece. The device includes a piercing member, such as a pin, which can be inserted into a selected aperture to break its respective seals. The configuration and movement of the pin are such that this action expels substantially no powder from the aperture.

Accordingly, in another preferred embodiment the invention relates to an inhalation device for dispensing single doses of a pharmaceutical composition in powder form from a container having a plurality of apertures, each of which holds a respective one of said doses, and is sealed by two opposed seals, the device comprising a housing for holding the container, the housing having an outlet and an airway which communicates with the outlet, and being configured to allow the container, to move relative thereto to bring each aperture in succession into registry with the airway, wherein the device includes a piercing member moveable from a retracted position in which it is positioned clear of the container into an extended position in which it extends through the aperture, said movement causing the piercing member to rapture the seals, and wherein the piercing member has a relatively small cross-sectional area compared with that of each aperture so that said movement of the piercing member expels substantially no medicament from the aperture, wherein the pharmaceutical composition comprises one or more, preferably one, anticholinergic.

In another preferred embodiment according to the invention the inhalation device according to WO 02/26302 is applied. The disclosure of WO 02/26302 is incorporated herein by reference in its entirety. WO 02/26302 describes an inhalation device for dispensing a pharmaceutical composition in powder form with an airway through which a dose travels from an ejection zone to an outlet of the airway. The airway has an inlet means which is so arranged as to create a jacket of air, flowing through a part of the airway extending from the ejection zone to the outlet. The jacket of air surrounds said dose and thereby prevents it form impinging on the airway walls. This reduces accumulation of material on the airway walls, and thus improves the consistency of performance of the inhalation device. Preferably, the inlet means includes a throat for producing a stream of fast flowing air which creates a zone of low pressure in front of the ejection zone, thereby to facilitate ejection of a dose.

Accordingly, in another preferred embodiment the invention relates to an inhalation device for dispensing doses of pharmaceutical composition in powder form, the device comprising an airway having an inlet means and an outlet through which the doses are dispensed, receiving means for receiving and retaining a dose of the pharmaceutical composition in an ejection zone in registry with the airway, from which ejection zone, in use, a dose of the pharmaceutical composition travels through the airway to the outlet, wherein the inlet means includes at least one inlet so positioned as to direct a flow of air into the airway at a region between a dose exiting the ejection zone and the wall of the airway, thereby providing, in use, a jacket of air, flowing from the ejection zone to the exit, which prevents particles of the ejected dose of the pharmaceutical composition from impinging on the airway walls, wherein the pharmaceutical composition comprises one or more, preferably one, anticholinergic.

In another preferred embodiment according to the invention the inhalation device according to WO 05/002654 is applied. The disclosure of WO 05/002654 is incorporated herein by reference in its entirety. WO 05/002654 describes an inhalation device for dispensing individual doses of a pharmaceutical composition in powder form from respective pockets of a disc-shaped carrier by outwardly rupturing a lidding foil by means of pressure on an opposite side surface, the device providing individual respective deaggregation flow paths for each pocket, split airstreams allowing improved entrainment of the pharmaceutical composition, a cam mechanism for outwardly rupturing the pockets, an indexing mechanism linked to the cam mechanism and a dose counter.

Accordingly, in another preferred embodiment the invention relates to an inhalation device for dispensing individual doses of a pharmaceutical composition in powder form from respective pockets of a carrier, the device including: a support for a carrier having a plurality of pockets containing respective doses of the pharmaceutical composition; and a mouthpiece through which to inhale an airstream carrying a dose of powder; the device further including: walls for defining individual respective first flow paths downstream of each respective pocket of a supported carrier wherein each individual respective first flow path is defined entirely by respective walls unique to that individual respective first flow path, is for connecting the corresponding respective pocket to the mouthpiece and is for deaggregating the powdered pharmaceutical composition in the airstream, wherein the pharmaceutical composition comprises one or more, preferably one, anticholinergic.

In another, particularly preferred embodiment according to the invention the inhalation device according to GB 2407042 and WO 2005/037353 is applied. The disclosures of GB 2407042 and WO 2005/037353 are incorporated herein by reference in their entirety. GB 2407042 and WO 2005/037353 describe an inhalation device that comprises a housing to receive a strip of blisters each having a puncturable lid and containing a dose of medicament for inhalation by a user, a mouthpiece through which a dose of medicament is inhaled by a user and, an actuator operable to sequentially move each blister into alignment with a blister piercing element. The actuator is also operable to cause the blister piercing element to puncture the lid of a blister such that, when the user inhales through the mouthpiece, an airflow through the blister is generated to entrain the dose contained therein and carry it out of the blister via the mouthpiece into the user's airway.

However, WO 2005/037353 discloses an inhalation device, which contains further features compared to the disclosure of GB 2407042. FIG. 30 of WO 2005/037353 discloses an inhalation device with a blister strip, the two ends of which are joined together. The blister strip, including the used blister elements, stay inside the device.

Accordingly, in another preferred embodiment the invention relates to an inhalation device, comprising a housing to receive a strip of blisters each having a puncturable lid and containing a dose of a pharmaceutical composition for inhalation by a user, a mouthpiece through which a dose of pharmaceutical composition is inhaled by a user and, an actuator operable to sequentially move each blister into alignment with a blister piercing element, said actuator also being operable to cause the blister piercing element to puncture the lid of a blister such that, when a user inhales through the mouthpiece, an airflow through the blister is generated to entrain the dose contained therein and carry it out of the blister and via the mouthpiece into the user's airway, wherein the pharmaceutical composition comprises one or more, preferably one, anticholinergic.

In another preferred embodiment the invention relates to the inhalation device mentioned hereinbefore, wherein the actuator is pivotally mounted to the housing. In another preferred embodiment the invention relates to the inhalation device mentioned hereinbefore, wherein the actuator comprises an arm pivotally mounted to the housing at one end.

In another preferred embodiment the invention relates to the inhalation device mentioned hereinbefore, wherein the blister piercing element depends from one side of said arm positioned so as to extend through an aperture in the housing in a closed position, in which the arm lies substantially against the housing, to pierce the lid of a blister aligned with the blister piercing element.

In another preferred embodiment the invention relates to the inhalation device mentioned hereinbefore, wherein the piercing element comprises at least two discrete piercing members operable to pierce a corresponding number of holes in a blister aligned with the blister piercing element.

In another preferred embodiment the invention relates to the inhalation device mentioned hereinbefore, wherein each piercing member comprises a central piercing blade and a pair of subsidiary piercing blades extending laterally across each end of the central piercing blade.

In another preferred embodiment the invention relates to the inhalation device mentioned hereinbefore, wherein the central piercing blade and the subsidiary piercing blades each have a pointed tip, the tip of the central piercing blade extending beyond the tips of each of the subsidiary piercing blades.

In another preferred embodiment the invention relates to the inhalation device mentioned hereinbefore, wherein an opening is formed in the arm in the vicinity of each piercing member, at least one of said openings s forming an airflow inlet into a blister and, at least one other of said openings forming an airflow outlet from a blister.

In another preferred embodiment the invention relates to the inhalation device mentioned hereinbefore, wherein the mouthpiece is on the arm and extends in a direction opposite to the direction in which the piercing members extend, the openings in the arm being in communication with the inside of the mouthpiece.

In another preferred embodiment the invention relates to the inhalation device mentioned hereinbefore, wherein the mouthpiece includes a primary chamber having an outside air inlet in communication, via the primary chamber, with the or each airflow inlet opening in the arm and, a secondary chamber in communication with the or each airflow outlet opening in said arm such that, when a user inhales through the mouthpiece, air is drawn through the or each airflow inlet opening into the blister via the outside air inlet and the primary chamber to entrain the dose in the airflow, said entrained dose passing through the or each airflow outlet openings into the secondary chamber of the mouthpiece from where it is carried into the user's airway.

In another preferred embodiment the invention relates to the inhalation device mentioned hereinbefore, wherein the primary and secondary chambers within the mouthpiece are separated by a partitioning wall.

In another preferred embodiment the invention relates to the inhalation device mentioned hereinbefore, wherein at least one air bypass aperture extends through the partitioning wall to communicate the primary chamber with the secondary chamber.

In another preferred embodiment the invention relates to the inhalation device mentioned hereinbefore, wherein the or each bypass aperture is configured such that the airflow from the primary chamber into the secondary chamber through the or each bypass aperture and the airflow from the or each airflow outlet openings meet substantially at right angles to each other.

In another preferred embodiment the invention relates to the inhalation device mentioned hereinbefore, comprising an indexing mechanism including an indexing member that moves so as to pull a blister into alignment with the blister piercing element. s

In another preferred embodiment the invention relates to the inhalation device mentioned hereinbefore, wherein the indexing member comprises an indexing wheel that rotates to move a blister into alignment with the blister piercing element.

In another preferred embodiment the invention relates to the inhalation device mentioned hereinbefore, wherein the indexing wheel is configured to rotate to move a blister into alignment with the blister piercing element in response to rotation of the actuator with respect to the housing in one direction, movement of the actuator in the same direction also being operable to puncture the lid of a blister aligned with the blister piercing element.

In another preferred embodiment the invention relates to the inhalation device mentioned hereinbefore, wherein the indexing wheel and the actuator include co-operating means thereon that engage when the actuator is rotated in one direction to cause rotation of the indexing wheel.

In another preferred embodiment the invention relates to the inhalation device mentioned hereinbefore, wherein the cooperating means comprises a set of ratchet teeth on the indexing wheel and a drive pawl on the actuator.

In another preferred embodiment the invention relates to the inhalation device mentioned hereinbefore, wherein the indexing wheel includes a plurality of recesses therein extending parallel to the axis of the wheel.

In another preferred embodiment the invention relates to the inhalation device mentioned hereinbefore, wherein the indexing wheel and actuator are coaxially mounted for rotation about the same axis.

In another preferred embodiment the invention relates to the inhalation device mentioned hereinbefore, including means to substantially prevent rotation of the indexing wheel other than by movement of the actuator in said one direction.

In another preferred embodiment the invention relates to the inhalation device mentioned hereinbefore, wherein said means comprises a first resiliently deformable anti-rotation pawl that extends into one of said recesses in the indexing wheel from the housing, the actuator including means for deflecting the first anti-rotation pawl from the recess to permit rotation of the indexing wheel when the drive pawl engages with the ratchet teeth.

In another preferred embodiment the invention relates to the inhalation device mentioned hereinbefore, wherein the actuator includes a drive plate and the means on the actuator for deflecting the first antirotation pawl comprises a release pin upstanding from the drive plate that engages with and resiliently deflects the pawl out of the recess to allow rotation of the indexing wheel.

In another preferred embodiment the invention relates to the inhalation device mentioned hereinbefore, wherein the means on the actuator comprises a second resiliently deformable anti-rotation pawl on the housing and a cam member on the actuator, the cam member engaging with a cam surface on the second anti-rotation pawl when the first antirotation pawl is deflected out of a recess to prevent rotation of the indexing wheel through more than a predetermined angle.

In another preferred embodiment the invention relates to the inhalation device mentioned hereinbefore, including a cap attached to the housing pivotable between a closed position in which it covers the actuator and mouthpiece and an open position in which the actuator and mouthpiece are revealed to enable a user to inhale through the mouthpiece.

In another preferred embodiment the invention relates to the inhalation device mentioned hereinbefore, wherein the indexing wheel rotates to move a blister into alignment with the blister piercing element in response to rotation of the cap with respect to the housing from the open to the closed position.

In another preferred embodiment the invention relates to the inhalation device mentioned hereinbefore, wherein the cap and the actuator include co-operating means to couple the actuator to the cap such that the actuator rotates relative to the housing in response to rotation of the cap between the open and closed positions.

In another preferred embodiment the invention relates to the inhalation device mentioned hereinbefore, wherein the cooperating means comprises a cam guide slot on the cap and a cam follower on the actuator slideably located within the cam guide slot.

In another preferred embodiment the invention relates to the inhalation device mentioned hereinbefore, wherein the cam guide slot is shaped such s that when the cap is rotated from its closed to its open position, the cam follower travels along the cam guide slot to rotate the actuator and cause the blister piercing element to pierce a blister aligned therewith and, when the cap is rotated from its open to its closed position, the cam travels back along the cam guide slot to cause the actuator to rotate in the opposite direction and withdraw the blister piercing element from the blister.

In another preferred embodiment the invention relates to the inhalation device mentioned hereinbefore, wherein the cam guide slot is configured so that the actuator does not rotate until towards the end of the movement of the cap from its closed to its open position and rotates at the beginning of the movement of the cap from its open to its closed position.

In another preferred embodiment the invention relates to the inhalation device mentioned hereinbefore, wherein the indexing wheel and the cap each include a toothed gear member mounted thereon engaged such that rotation of the cap between the open and closed positions causes rotation of the gear member on the indexing wheel.

In another preferred embodiment the invention relates to the inhalation device mentioned hereinbefore, wherein a clutch member couples the gear member on the indexing wheel to the indexing wheel such that the indexing wheel rotates together with the gear member coupled thereto when the cap is rotated from the open to the closed position to move a subsequent blister into alignment with the blister piercing element.

In another preferred embodiment the invention relates to the inhalation device mentioned hereinbefore, wherein the housing includes a chamber to receive used blisters.

In another preferred embodiment the invention relates to the inhalation device mentioned hereinbefore, wherein the chamber is covered by a lid attached to the housing which is openable to facilitate removal of a portion of used blisters from the blisters remaining in the device.

In another preferred embodiment the invention relates to the inhalation device mentioned hereinbefore, including a separating element on the housing operable to enable detachment of said portion of used blisters.

In another preferred embodiment the invention relates to the inhalation device mentioned hereinbefore, wherein the separating element includes a resilient blister grip which is operable to press a blister strip against the housing to facilitate separation of said portion from said remaining blisters.

In another preferred embodiment the invention relates to the inhalation device mentioned hereinbefore, incorporating a coiled strip of blisters, each having a puncturable lid and containing a dose of medicament for inhalation by a user, located in the housing.

In another preferred embodiment the invention relates to the inhalation device mentioned hereinbefore, wherein the strip includes a frangible cut line between each blister to facilitate detachment of a blister from an adjacent blister along said line.

In another preferred embodiment the invention relates to the inhalation device mentioned hereinbefore, wherein the strip includes a notch to facilitate tearing of the strip between each blister.

In another preferred embodiment the invention relates to the inhalation device mentioned hereinbefore, wherein the coiled strip carries between 30 and 60 blisters and each blister has a dose payload of between 10 and 20mg.

In another preferred embodiment the invention relates to the inhalation device mentioned hereinbefore, formed from no more than four moulded components.

In another preferred embodiment the invention relates to the inhalation device mentioned hereinbefore, formed from no more that five moulded components.

In another preferred embodiment the invention relates to the inhalation device mentioned hereinbefore, formed from no more than six moulded components.

In another preferred embodiment the invention relates to the inhalation device mentioned hereinbefore, wherein the housing is wholly or partially formed from a transparent material.

The inhalation device according to GB 2407042, is of interest. Specific embodiments of the said device are illustrated in GB 2407042 for instance by FIGS. 1 to 19.

FIG. 1 according to GB 2407042 is a perspective view of an inhaler according to an embodiment of the invention;

FIG. 2 according to GB 2407042 is a perspective view of the inhaler illustrated in FIG. 1 with the cap open to reveal the mouthpiece and the actuator in a closed position;

FIG. 3 according to GB 2407042 is a perspective view of the inhaler illustrated in FIG. 2 according to GB 2407042 with the actuator in an open position;

FIG. 4 according to GB 2407042 is a perspective view of the inhaler shown in FIG. 1 according to GB 2407042 with a used blister chamber cover open;

FIG. 5 according to GB 2407042 is an exploded perspective view of the inhaler illustrated in FIGS. 1 to 4 according to GB 2407042 also showing a coiled strip of blisters used with the device according to the invention;

FIG. 6 according to GB 2407042 is a rear cross-sectional view of the inhaler illustrated in FIGS. 1 to 5 according to GB 2407042 with the actuator shown separately;

FIG. 7 according to GB 2407042 is a front cross-sectional view of the inhaler illustrated in FIG. 6 according to GB 2407042 in which the actuator is pivotally mounted to the housing;

FIGS. 8A and 8B according to GB 2407042 shows the configuration of the piercing elements on the actuator and a small portion of a strip of blisters to illustrate the type of cut made therein by the piercing elements, respectively;

FIG. 9 according to GB 2407042 is a side sectional view of the mouthpiece and actuator during inhalation from a blister;

FIGS. 10A to 10C according to GB 2407042 show a series of front cross-sectional views of the inhaler according to the invention with a blister strip located therein to show the path of used blisters from the housing;

FIG. 11 according to GB 2407042 is an exploded side cross-sectional view of an inhaler according to another embodiment of the invention;

FIGS. 12A and 12B according to GB 2407042 are side cross-sectional views of the inhaler according to the second embodiment with the cap in the closed and open positions respectively;

FIG. 13 according to GB 2407042 shows a short portion of a strip of blisters for use in the inhaler according to any embodiment of the invention;

FIGS. 14A and 14B according to GB 2407042 are perspective views of another embodiment of inhaler according to the present invention;

FIGS. 15A and 15B according to GB 2407042 show a side cross-sectional view of the inhaler illustrated in FIGS. 14A and 14B according to GB 2407042 with the actuator in a closed and open position respectively.

FIG. 16 according to GB 2407042 is another side cross-sectional view of the inhaler shown in FIGS. 14A and 14B according to GB 2407042;

FIG. 17 according to GB 2407042 is a side sectional view of the mouthpiece and actuator during inhalation from a blister;

FIG. 18 according to GB 2407042 shows an alternative configuration of piercing elements on the actuator according to any embodiment of the invention, and

FIG. 19A according to GB 2407042 shows the airflow into the blister using the piercing elements of FIG. 8A according to GB 2407042 and

FIG. 19B according to GB 2407042 shows the airflow into the blister using the piercing element of FIGS. 18-11 according to GB 2407042.

Within the disclosure of the instant invention the term pharmaceutical compositions in powder form may also be replaced by one or several of the terms pharmaceutical composition, powder, inhalable powder or the like. It is to be understood that the instant invention is exclusively directed to powdered compositions suitable for inhalation. The pharmaceutical compositions in powder form may consist of an anticholinergic, optionally in combination with a betamimetic or steroid, on their own or of a mixture of the these active substances with pharmaceutically acceptable excipients. If the active substances are present in admixture with pharmaceutically acceptable excipients, preferably the pharmaceutically acceptable excipients mentioned hereinbefore may be used.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronisedanticholinergic, and optionally one or more betamimetic and/orsteroid, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art.

For the methods of preparing the pharmaceutical compositions in powder form reference may be made to the disclosure of WO 02/30390, WO 03/017970, or WO 03/017979 for example. The disclosure of WO 02/30390, WO 03/017970, and WO 03/017979 is herby incorporated by reference into the instant patent application in its entirety.

As an example, the pharmaceutical compositions according to the invention may be obtained by the method described below.

First, the excipient and the active substance are placed in a suitable mixing container. The active substance used has an average particle size of 0.5 to 10 μm, preferably 1 to 6 μm, most preferably 2 to 5 μm. The excipient and the active substance are preferably added using a sieve or a granulating sieve with a mesh size of 0. 1 to 2 mm, preferably 0.3 to 1 mm, most preferably 0.3 to 0.6 mm. Preferably, the excipient is put in first and then the active substance is added to the mixing container. During this mixing process the two components are preferably added in batches. It is particularly preferred to sieve in the two components in alternate layers. The mixing of the excipient with the active substance may take place while the two components are still being added. Preferably, however, mixing is only done once the two components have been sieved in layer by layer.

If after being chemically prepared the active substance used in the process described above is not already obtainable in a crystalline form with the particle sizes mentioned earlier, it can be ground up into the particle sizes which conform to the above-mentioned parameters (so-called micronising).

1. An inhalation device comprising a plurality of doses of a pharmaceutical composition in powder form, wherein the pharmaceutical composition comprises at least one anticholinergic, optionally in combination with a pharmaceutically acceptable excipient, wherein the anticholinergic is selected from the group consisting of
a) tiotropium salts,
b) compounds of formula 1c 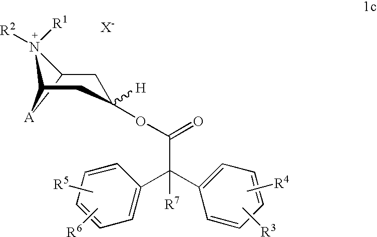
wherein
A denotes a double-bonded group selected from among 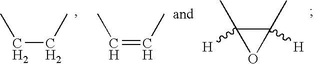
X⁻ denotes an anion with a single negative charge, preferably an anion selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹ and R² which may be identical or different denote a group selected from among methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂-CH₂—F, —O—CH₂—F, —O—CH₂-CH₂—F, —CH₂—OH, —CH₂-CH₂—OH, CF₃, —CH₂—OMe, —CH₂-CH₂—OMe, —CH₂—OEt, —CH₂-CH₂—OEt, —O—COMe, —O—COEt, —O—COCF₃, —O—COCF₃, fluorine, chlorine or bromine;
c) compounds of formula 1d 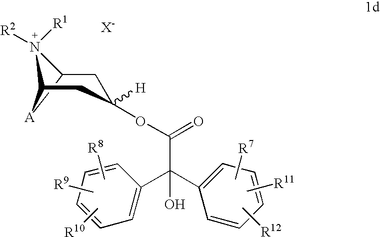
wherein
A, X⁻, R¹ and R² may have the meanings as mentioned hereinbefore and wherein
R⁷, R⁸, R⁹, R¹⁰, R¹¹ and R¹² , which may be identical or different, denote hydrogen, methyl, ethyl, methyloxy, ethyloxy, hydroxy, fluorine, chlorine, bromine, CN, CF₃ or NO₂, with the proviso that at least one of the groups R⁷, R⁸, R⁹, R¹⁰, R¹¹ and R¹² is not hydrogen,
d) compounds of formula 1e 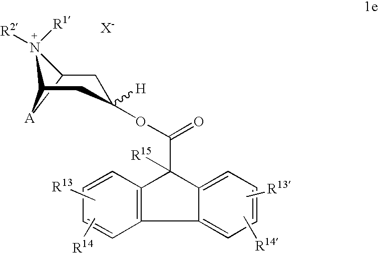
wherein A and X⁻ may have the meanings as mentioned hereinbefore, and wherein
R¹⁵ denotes hydrogen, hydroxy, methyl, ethyl, —CF₃, CHF₂ or fluorine;
R^{1′} and R^{2′} which may be identical or different denote C₁-C₅-alkyl which may optionally be substituted by C₃-C₆-cycloalkyl, hydroxy or halogen, or
R^{1′} and R^{2′} together denote a —C₃-C₅-alkylene-bridge;
R¹³, R¹⁴, R^{13′} and R^{14′} which may be identical or different denote hydrogen, —C₁-C₄-alkyl, —C₁-C₄-alkyloxy, hydroxy, —CF₃, —CHF₂, CN, NO₂ or halogen,
e) compounds of formula 1f 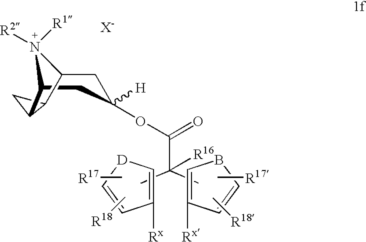
wherein X⁻ may have the meanings as mentioned hereinbefore, and wherein
D and B which may be identical or different, preferably identical, denote —O, —S, —NH, —CH₂, —CH═CH, or —N(C₁-C₄-alkyl)-;
R¹⁶ denotes hydrogen, hydroxy, —C₁-C₄-alkyl, —C₁-C₄-alkyloxy, —C₁-C₄-alkylene-Halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC—C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or
R^{1″} and R^{2″} together denote a —C₃-C₅-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂ or halogen;
R^{x} and R^{x′} which may be identical or different, denote hydrogen, C₁-C₄-alkyl, C₁-C₄-alkyloxy, hydroxy, —CF₃, —CHF₂, CN, NO₂ or halogen or
R^{x} and R^{x′} together denote a single bond or a bridging group selected from among the bridges —O, —S, —NH, —CH₂, —CH₂-CH₂—,
N(C₁-C₄-alkyl), —CH(C₁-C₄-alkyl)- and —C(C₁-C₄-alkyl)₂, and
f) compounds of formula 1g 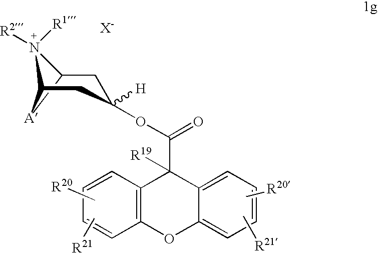
wherein X⁻ may have the meanings as mentioned hereinbefore, and wherein
A′ denotes a double-bonded group selected from among 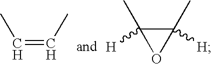
R¹⁹ denotes hydroxy, methyl, hydroxymethyl, ethyl, —CF₃, CHF₂ or fluorine;
R^{1′″} and R^{2′″} which may be identical or different denote C₁-C₅-alkyl which may optionally be substituted by C₃-C₆-cycloalkyl, hydroxy or halogen, or
R^{1′} and R^{2′″} together denote a —C₃-C₅-alkylene-bridge;
R²⁰, R²¹, R^{20′} and R^{21′} which may be identical or different denote hydrogen, —C₁-C₄-alkyl, —C₁-C₄-alkyloxy, hydroxy, —CF₃, —CHF₂, CN, NO₂ or halogen.

2. An inhalation device in accordance with claim 1 comprising at least one active pharmaceutical ingredient in addition to the at least one anticholinergic.

3. An inhalation device in accordance with claim 1 further comprising at least one betamimetic.

4. An inhalation device in accordance with claim 1 further comprising at least one steroid.

5. An inhalation device in accordance with claim 1 further comprising at least one betamimetic and at least one steroid.

6. An inhalation device in accordance with claim 2 wherein the at least one active pharmaceutical ingredient in addition to the at least one anticholinergic is a beta₂ agonist selected from the group consisting of albuterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol and the pharmacologically acceptable acid addition salts thereof.

7. An inhalation device in accordance with claim 2 wherein the at least one active pharmaceutical ingredient in addition to the at least one anticholinergic is a betamimetic selected from the group consisting of fenoterol, formoterol, salmeterol, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, and the pharmacologically acceptable acid addition salts thereof.

8. An inhalation device in accordance with claim 2 wherein the at least one active pharmaceutical ingredient in addition to the at least one anticholinergic is a steroid selected from the group consisting of prednisolone, prednisone, butixocortpropionate, RPR-106541, flunisolide, beclomethasone, triamcinolone, budesonide, fluticasone, mometasone, ciclesonide, rofleponide, ST-126, dexamethasone, (S)-fluoromethyl 6α,9 α-difluoro-17α-[(2-furanylcarbonyl)oxy]-11β-hydroxy-16α-methyl-3-oxo-androsta-1,4-diene-17β-carbothionate, (S)-(2-oxo-tetrahydro-furan-3S-yl)6α,9 α-difluoro-11β-hydroxy-16α-methyl-3-oxo-17α-propionyloxy-androsta-1,4-diene-17β-carbothionate, and etiprednol-dichloroacetate (BNP-166).
